(19) United States
(12) Patent Application Publication
(10) Pub. No.: US 2008/0086158 A1
(43) Pub. Date: Apr. 10, 2008

(54) SCALPEL WITH PLASTIC BLADE CARRIER, HANDLE, OR BOTH

(75) Inventors: Robert Gerard Holman, Johannesburg (ZA); Edwin Marcus Letty, Sandton (ZA)

Correspondence Address: OCCHIUTI ROHLICEK & TSAO, LLP, 10 FAWCETT STREET, CAMBRIDGE, MA, 02138, US

(73) Assignee: Trevor John Milton, Johannesburg (ZA)

(21) Appl. No.: 11/867,929

(22) Filed: Oct. 5, 2007

(30) Foreign Application Priority Data
Oct. 6, 2006 (ZA) 2006/08336

(51) Int. Cl. A61B 17/32

(52) U.S. Cl. 606/167

(57) ABSTRACT

A scalpel is provided having a handle, a plastic blade carrier, and a scalpel blade secured to the blade carrier. The blade carrier is preferably sufficiently transparent to enable at least the shape of a blade secured to a flat face thereof to be visible from the opposite side of the blade carrier. Preferably, the blade carrier is made of a clear plastic material that may be colourless or tinted. The scalpel blade may be a fixed scalpel blade in which the blade carrier is integral with the handle. Alternatively, the scalpel blade may be retractable into a hollow handle that is also preferably sufficiently transparent to enable at least the shape of the scalpel blade to be visible through the wall of the handle.

### FIELD OF THE INVENTION

This invention relates to a scalpel having a plastic blade carrier, handle, or both wherein the scalpel blade is fixed to a plastic blade carrier. More particularly, but not exclusively, the invention relates to disposable scalpels.

### BACKGROUND TO THE INVENTION

Disposable scalpels are produced in large numbers in basically one of two different forms; a more sophisticated one in which a retractable scalpel blade is fixed to a blade carrier that is longitudinally movable within a hollow handle; and a less sophisticated one in which the scalpel blade is fixed directly to the handle that also forms the blade carrier.

In either instance, the scalpel blade has a mounting base that has one or more apertures through it and that is firmly clamped to a flat face of the blade carrier typically by means of one or more integral plastics protrusions that pass through the apertures and are thermally deformed to lock the blade permanently in position on the blade carrier.

Of necessity, the portion of the blade carrier to which the mounting base of the blade is secured is of significant size for purposes of strength and may accordingly obscure the scalpel blade, at least to some extent. This is particularly so when the scalpel is viewed from the side thereof opposite the flat face of the blade carrier. Whilst the visibility of the blade may be satisfactory when viewed from the side of the handle corresponding to the flat face of the blade carrier, the visibility of the blade may not be satisfactory from the opposite side. Thus, left-handed and right-handed use by surgeons will result in quite different views of the scalpel blade that is being used. This results from the invariably opaque nature of the plastics blade carrier.

Also, in the case of retractable scalpel blades, it is not always apparent that the blade has been retracted adequately or, in the event that two or more retracted positions are possible, which of the retracted positions applies at any particular time. This also results from the fact that the handles of scalpels having retractable blades are invariably opaque.

### OBJECT OF THE INVENTION

It is one object of this invention to provide a scalpel having a scalpel blade fixed to a plastic carrier in which visibility of the blade is enhanced.

It is another object of this invention provide a scalpel having a retractable blade wherein the retracted position of the blade can be more easily determined.

### SUMMARY OF THE INVENTION

In accordance with one aspect of this invention there is provided a scalpel having a handle, a plastic blade carrier, and a scalpel blade secured to the blade carrier,

wherein the blade carrier is sufficiently transparent to enable at least the shape of a blade secured to a flat face thereof to be visible from the opposite side of the blade carrier.

A further feature of the invention provides for the blade carrier to be made of a clear plastic material that is preferably colourless, but is optionally tinted.

In the instance of a scalpel having a scalpel blade that is retractable into a hollow handle, the longitudinally movable blade carrier is made to be sufficiently transparent as aforesaid.

In the instance of a scalpel having a fixed scalpel blade, the blade carrier forms part of, and is integral with, the handle with at least the carrier portion being sufficiently transparent as aforesaid.

In accordance with a second aspect of the invention there is provided a scalpel having a scalpel blade mounted to a carrier therefor that is longitudinally slidable relative to a hollow handle such that the scalpel blade can be selectively retracted to an inoperative position within the handle or extended from one end of the handle to an operative position,

wherein the hollow handle is sufficiently transparent to enable at least the shape of the scalpel blade to be visible through the wall of the handle.

In order that the above and other features of the invention may be more fully understood different embodiments thereof will now be described with reference to the accompanying drawings.

### BRIEF DESCRIPTION OF THE DRAWINGS

In drawings:—

FIG. 1 is a side view from one side of a scalpel having a retractable blade;

FIG. 2 is a side view from the opposite side thereof illustrating the conventional arrangement of an opaque blade carrier;

FIG. 3 is a view similar to FIG. 2 but illustrating a transparent blade carrier according to the invention;

FIG. 4 is a side view of a scalpel having a retractable scalpel blade with the blade in the retracted condition and showing the handle formed according to the second aspect of this invention;

FIG. 5 is a side view from one side of a scalpel having a fixed scalpel blade and wherein the handle and integral blade carrier are formed according to the invention; and,

FIG. 6 is a side view from the opposite side of the scalpel illustrated in FIG. 5.

### DETAILED DESCRIPTION WITH REFERENCE TO THE DRAWINGS

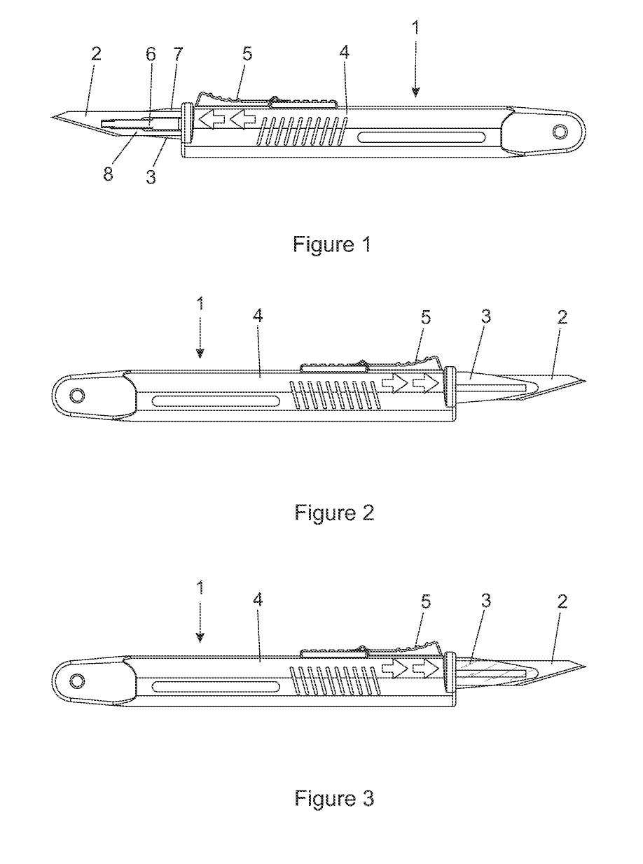

Referring firstly to FIGS. 1 and 2 of the drawings, there is illustrated a scalpel, generally indicated by numeral (1), having a retractable scalpel blade (2) fixed, in the usual way, to an injection moulded plastics blade carrier (3) movable longitudinally within an injection moulded plastics handle (4) by means of a manually operable slider (5). The position of the slider on the handle is irrelevant to the invention and it may be at the top edge, as illustrated, or it could be on either side of the handle.

The blade carrier has one or more integral plastics protrusions (6) in the central region of a flat face (7) against which a mounting base (8) of the blade is fixed by thermally derforming the protrusions. In order to provide required rigidity to the blade, the blade carrier is fairly substantial and its peripheral shape extends beyond the periphery of the mounting base of the blade.

The situation in the instance that the blade carrier is made of conventional opaque plastics material is illustrated most clearly in FIG. 2 in which a substantial part of the blade is obscured.

However, as provided by this invention, the blade carrier is made of a transparent, and preferably a colourless plastics material in which instance, as illustrated in FIG. 3, the outside of the entire scalpel blade can be observed from the side of the blade carrier opposite the flat face to which the blade is secured. The blade carrier may optionally be tinted to a desired colour, if required

Accordingly, a surgeon using the scalpel in either hand has enhanced visibility of the scalpel blade.

It will be appreciated that the concept described above can be applied to any scalpel having a plastics blade carrier. Thus, as illustrated in FIGS. 5 and 6, the invention can equally well be applied to a scalpel having a scalpel blade (9) permanently fixed directly to an injection moulded plastics handle (10) in a similar manner to that described above. In this instance, the entire handle is made of a transparent plastics material, typically colourless, but also optionally tinted to a required colour.

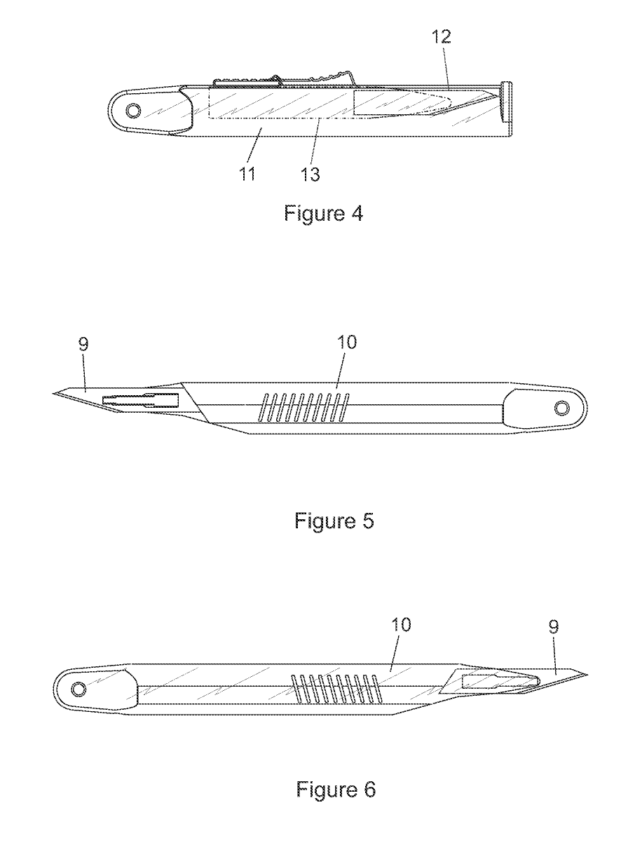

Referring now to FIG. 4 of the drawings, there is illustrated an embodiment of the second aspect of the invention in which the handle (11) of a scalpel having a retractable blade is made transparent so that the retracted blade (12) can be viewed through the wall of the handle and its position within the handle determined. This position may be a fully retracted position, a partially retracted position, or a finally locked and immobilised innermost position in which the scalpel is rendered non-reusable.

Another advantage of this aspect of the invention is in that the size and configuration of the blade can be viewed through the transparent handle wall. This obviates the necessity for having to have knowledge or reference material that would correlate the usual colour coding of the slider with the size and shape of blade.

Clearly, the blade carrier (13) in the instance of the embodiment of the second aspect of the invention would generally also be made transparent in terms of the first aspect of the invention.

It will be understood that numerous variations may be made to the embodiment of the invention described above without departing from the scope hereof.

1. A scalpel having a handle, a plastic blade carrier, and a scalpel blade secured to the blade carrier,
wherein the blade carrier is sufficiently transparent to enable at least the shape of a blade secured to a flat face thereof to be visible from the opposite side of the blade carrier.

2. A scalpel as claimed in claim 1 in which the blade carrier is made of a clear plastic material.

3. A scalpel as claimed in claim 2 in which the clear plastic material is colourless.

4. A scalpel as claimed in claim 2 in which the clear plastic material is tinted.

5. A scalpel as claimed in claim 1 in which the scalpel blade is a fixed scalpel blade and the blade carrier forms part of, and is integral with, the handle with at least the carrier portion being sufficiently transparent as aforesaid.

6. A scalpel as claimed in claim 1 in which the handle is hollow and the scalpel blade is retractable into the hollow handle.

7. A scalpel as claimed in claim 6 in which the hollow handle is sufficiently transparent to enable at least the shape of the scalpel blade to be visible through the wall of the handle.

8. A scalpel having a scalpel blade mounted to a blade carrier therefor that is longitudinally slidable relative to a hollow handle such that the scalpel blade can be selectively retracted to an inoperative position within the handle or extended from one end of the handle to an operative position,
wherein the hollow handle is sufficiently transparent to enable at least the shape of the scalpel blade to be visible through the wall of the handle.
